(19) United States
(12) Patent Application Publication
(10) Pub. No.: US 2021/0177514 A1
(43) Pub. Date: Jun. 17, 2021

(54) THERAPEUTIC MICROWAVE ABLATION DEVICES AND METHODS

(71) Applicant: Kammio, Inc., San Jose, CA (US)

(72) Inventors: Raj Subramaniam, Fremont, CA (US); Jessi Johnson, Sunnyvale, CA (US); Mariam H. Taimisto, San Jose, CA (US)

(21) Appl. No.: 16/953,901

(22) Filed: Nov. 20, 2020

Related U.S. Application Data
(60) Provisional application No. 62/937,853, filed on Nov. 20, 2019.
(60) Provisional application No. 62/937,822, filed on Nov. 20, 2019.

(51) Int. Cl. A61B 18/18; H01P 3/10; A61B 5/318; A61B 5/00

(52) U.S. Cl. CPC A61B 18/1815; H01P 3/10; A61B 2018/00577; A61B 5/4836; A61B 5/318

(57) ABSTRACT

A device for treating tissue has a plurality of conductors that form one or more transmission lines configured to deliver microwave energy to target tissue. In addition to transmission lines, the device also may include an anchor element used to penetrate the target ablation tissue and anchor the device. A system includes the device, a microwave generator, and a coaxial cable. A biocompatible solution may be injected into the target ablation region to alter tissue properties or to facilitate visualization of the ablation region.

The present application is a non-provisional of, and claims the benefit of US Provisional Patent Application Nos. 62/937,822 (Attorney Docket No. 5191.001PV3) filed Nov. 20, 2019; and 62/937,853 (Attorney Docket No. 5192.001PV3) filed Nov. 20, 2019; the entire contents of each is incorporated herein by reference.

### BACKGROUND

Microwaves are a form of electromagnetic radiation with wavelengths broadly ranging from about one meter to one millimeter, with frequencies ranging between 300 MHz (1 m) and 300 GHz (1 mm). A more common definition is the range between 1 and 100 GHz (wavelengths between 0.3 m and 3 mm).

Microwaves are widely used in modern technology, for example in communication links, wireless networks, microwave relay networks, radar, satellite and space communications, cooking food, etc.

Microwave technology has been used as an energy source in many medical devices. Due to several technical and practical reasons, microwave devices are not as common as the devices that use radiofrequency current as the energy source.

Additional information about the use of microwave energy is disclosed in Compilation of the Dielectric Properties of Body Tissues at RF and Microwave Frequencies, Report N.AU/OE-TR-1996-037, Occupational and environmental health directorate, Radiofrequency Radiation Division, Brooks Air Force Base, Tex. (USA), 1996 by C. Gabriel; the entire contents of which are incorporated herein by reference.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a two-wire transmission line.

FIG. 2 shows E-field vectors along a two-wire transmission line.

FIGS. 3A-3C illustrate power absorption patterns for a two-wire transmission line.

FIGS. 4A-4C illustrate electric field patterns for a two-wire transmission line.

FIGS. 5A-5C show power absorption plots corresponding to the E-field patterns in FIGS. 4A-4C.

FIGS. 6A-6B show another example of a two-wire line.

FIG. 7 shows S11 vs. frequency for a coaxial fed two wire transmission line.

FIG. 8 shows another plot of S11 vs. frequency.

FIG. 9 shows still another plot of S11 vs. frequency.

FIG. 10 shows yet another plot of S11 vs. frequency.

FIGS. 11A-1C show a three-wire transmission line with corresponding E-field and absorption pattern.

FIGS. 12A1-12A5 show various configurations of wire transmission lines.

FIGS. 12B1-12B3 show various configurations of wire transmission lines.

FIGS. 13A-13B show a two-wire transmission line and corresponding S11 vs. frequency plot.

FIG. 14 shows an absorption pattern.

FIGS. 15A-15B show a loop transmission line and ablation boundary.

FIG. 16 shows a two-wire transmission line.

FIG. 17 shows a two-wire transmission line.

FIG. 18 shows another two-wire transmission line.

FIGS. 19A-19B show a looped transmission line and corresponding magnetic field pattern.

FIG. 20 shows dual coaxial conductor needles.

FIGS. 21A-21C show a dual coaxial needle example with absorption patterns.

FIGS. 22A-22C show a microwave applicator example with sheets.

FIGS. 23A-23B show a PCB based loop applicator example and lesion pattern.

FIGS. 24A-24C show an example of anchoring a microwave ablation device to tissue.

FIGS. 25-26 show examples of moveable needles.

FIG. 27 shows an example cardiac ablation device.

FIG. 28 shows a plot of S11 vs. frequency of the example in FIG. 27.

FIG. 29 shows an example of a three-wire transmission device.

FIGS. 30A-30B show an example of 4-needle microwave transmission probe.

FIG. 31 illustrates an example of a 4-needle microwave probe.

FIG. 32 shows an example of transmission device formed on a printed circuit board.

FIG. 33 shows a transmission device formed on a printed circuit board disposed in tissue.

FIG. 34 shows S11 vs. frequency of the tumor ablation design in FIG. 33

FIG. 35A shows an example of a device with three traces forming a closed loop.

FIG. 35B shows the ablation boundary of the device in FIG. 35A.

FIGS. 36A-36B show an example of a 3-wire transmission line and ablation boundary.

FIG. 37 shows a plot of dB magnitude vs. frequency.

FIG. 38 shows an example of a four-needle device.

FIG. 39 shows a plot of S11 vs. frequency for the device in FIG. 38.

FIG. 40 shows S11 dB is minimized when dielectric constant is reduced.

FIGS. 41A-41B show examples of filter circuits.

### DETAILED DESCRIPTION

Microwave technology has been used as an energy source in many medical devices. Due to several technical and practical reasons, microwave devices are not as common as the devices that use radiofrequency (RF) current as the energy source. However, microwave technology may offer some unique advantages over RF devices such as avoiding dangerous steam pops in cardiac, tumor or other tissue ablation or may be largely immune to desiccation experienced around RF electrodes which can cause large increases in resistance which deteriorate the ability of RF to deliver power to tissue. At least some of these challenges may be overcome by using microwave energy in a therapeutic procedure using at least some of the examples disclosed herein.

The present examples are novel device configurations that transmit the microwave energy deep into the tissue to create large lesions. This can be very useful in medicine where control of large lesions is necessary to treat an illness. Similarly, the present examples may be used to treat tumors (benign and malignant) in various parts of the human anatomy. In general, the examples described herein can be used to selectively kill or denature targeted tissue to bring about a therapeutic change. The change effected may be controllably varied to bring partial damage to regulate a bodily function or response such as neuromodulation, or may remodel the tissue in shape, volume, or another characteristic.

Microwave energy offers some unique advantages over the RF devices. At least one of the advantages of the microwave is its ability to penetrate deeper into the tissue. With radiofrequency ablation, most of the resistive heating is confined to about a millimeter of the tissue. Thermal propagation is the only pathway for the RF lesion to grow. When deeper lesions are desired, this can cause a significant problem. Often the tissue in contact with the RF electrode is desiccated. In the worst case, steam pops create an explosion of the tissue with dangerous consequences. With proper design and correct frequency, microwave energy can penetrate and heat a large volume of the tissue at the same or lower power levels compared to RF. Due to this phenomenon, the thermal footprint of the lesion grows faster and safer with microwave treatment.

Open-Ended Dual Needle Applicator: Two Wire Transmission Line

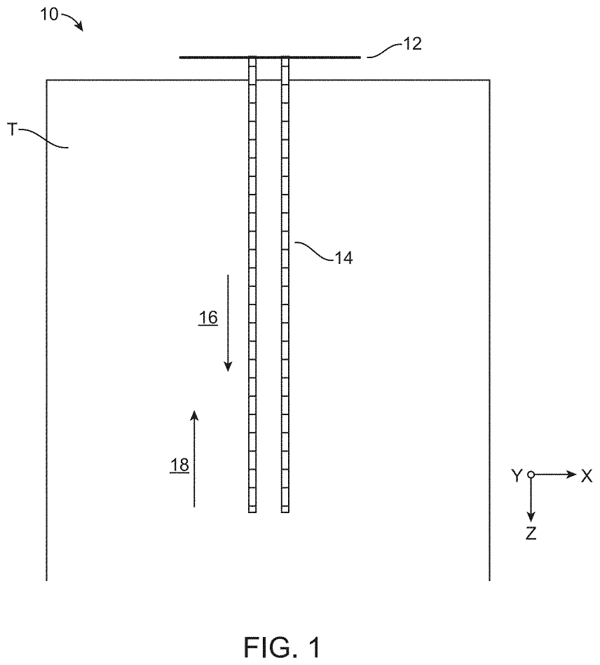

An ideal two wire transmission line 10 is illustrated in FIG. 1. The ideal two wire transmission line 10 may be disposed in muscle-like dielectric material T. An incident microwave signal is launched into the ideal input port 12. The signal propagates down 16 the transmission line, strikes the open end and reflects 18 toward the input port 12, causing a standing wave pattern along the line. In addition to the standing wave effect, power is deposited into tissue as the wave is propagating down the line. Length of the transmission line and frequency can be utilized to create a desired absorption/heating pattern. Here, the two-wire transmission line may be formed from any two conductors such as wire filaments or needles 14 with an optional tissue piercing tip

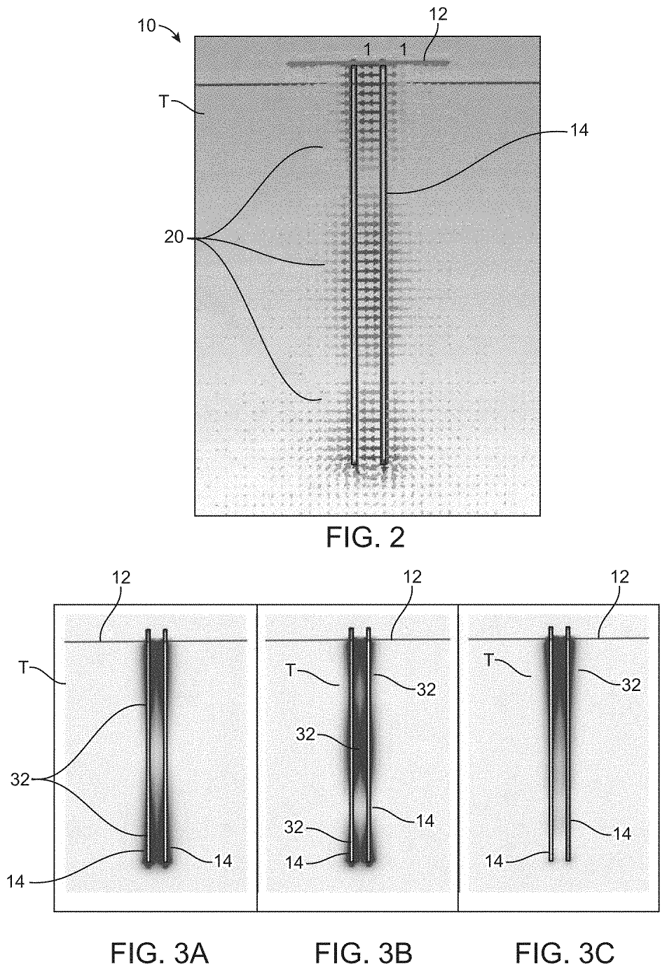

FIG. 2 shows a plot of E-field vectors along the two-wire transmission line illustrated in FIG. 1, at a frequency of 2 GHz. E-field 20 is focused between the two lines 14, which can be utilized to create a large absorption zone between the conductors which in this example may be two needles. The regions of intense E-field may be aligned horizontally between the two transmission lines

FIGS. 3A-3C illustrate examples of power absorption patterns (scaled to 1/10 of max) for two wire transmission line needles 14 in a block of muscle tissue T (real part of permittivity (er)=53.5, imaginary part of permittivity (ei)=12.9 @2 GHz). Black/dark coloring near the wires indicates regions of highest absorption. The non-limiting wire parameters in this example are: diameter=0.287 mm, wire spacing=1.5 mm, and wire length=20 mm.

FIG. 3A shows the region of highest absorption may be in two locations 32 along the transmission lines 14 where high absorption occurs at a proximal portion of the transmission lines and a distal portion of the transmission lines. Here, the frequency=1 GHz, and the standing wave pattern creates constructive peaks at the proximal and distal end of the needles.

FIG. 3B shows another example where the region of highest absorption may be in three locations 32 along the transmission lines 14 where high absorption occurs at a proximal portion of the transmission lines, a distal portion of the transmission lines and a region in between the proximal portion of the transmission lines and the distal portion of the transmission lines. Here, the frequency=2 GHz, and the standing wave pattern creates constructive peaks at the proximal and distal ends of the needles, as well as at the center of the needles.

FIG. 3C shows another example where the region of highest absorption 32 is in a proximal portion of the transmission lines 14. In another example, the region of highest absorption is in a distal portion of the transmission, or the region of highest absorption is between the distal portion and the proximal portion of the transmission lines. Here, the frequency=5.8 GHz, and in this case the losses along the transmission line are so large that the energy exists mainly in the proximal end of the needles.

Controlling the location of constructive peaks may be advantageous in the case where a product is supplied in a single length needle electrode (e.g. 20 mm), but the area of the energy delivery is only required at a depth of 5 mm. In this case, the needles can penetrate through the tissue to the full length of 20 mm, but the operator choose the 5.8 GHz frequency to limit the lesion depth.

Other aspects of the device in FIGS. 3A-3C may be substantially the same as discussed in FIGS. 1-2 above.

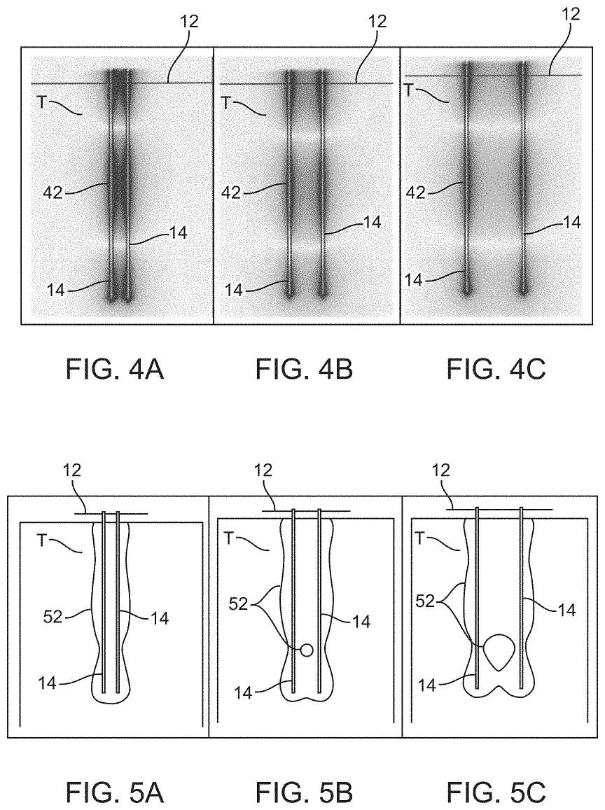

FIGS. 4A-4C shows example Electric Field (E-field) patterns 42 for 20 mm long needles 14 at 2 GHz, with all plots normalized to the same peak E-field value. Smaller spacing between needles 14 has more intense E-field as indicated by the darker color around the electrode needles 14.

FIG. 4A shows needle spacing=1.5 mm.

FIG. 4B shows needle spacing=3.0 mm.

FIG. 4C shows needle spacing=5.0 mm.

The distance between the two wires, needles or other conductors determines how intense of an E-field exists between them. Closer proximity leads to stronger E-fields and a higher absorption level as seen in FIG. 4A. This may be used for a more narrow but efficient ablation pattern. A larger distance between needles decreases the E-field but spreads the energy over a larger volume which may be used for creating larger lesions as seen in FIG. 4C. FIG. 4B illustrates an intermediate example where E-field and volume are between the examples in FIGS. 4A and 4C.

Other aspects of FIGS. 4A-4C may be substantially the same as discussed in FIGS. 1-2 above.

FIGS. 5A-5C show power absorption plots 52 corresponding to the E-field patterns in the previous figures (FIGS. 4A-4C) where FIG. 5A corresponds with FIG. 4A, and FIG. 5B corresponds with FIG. 5B, etc. The outline 52 around and between the electrodes which may be needles or other conductor elements denote regions of equivalent relative energy absorption for each configuration. This demonstrates an increased treatment zone for needles 14 with further separation.

FIG. 5A shows needle spacing=1.5 mm.

FIG. 5B shows needle spacing=3.0 mm.

FIG. 5C shows needle spacing=5.0 mm.

Other aspects of FIGS. 5A-5C may be substantially the same as in FIGS. 1-2 above.

Open-Ended Dual Needle Applicator: Two Wire Line Fed with Coaxial Cable

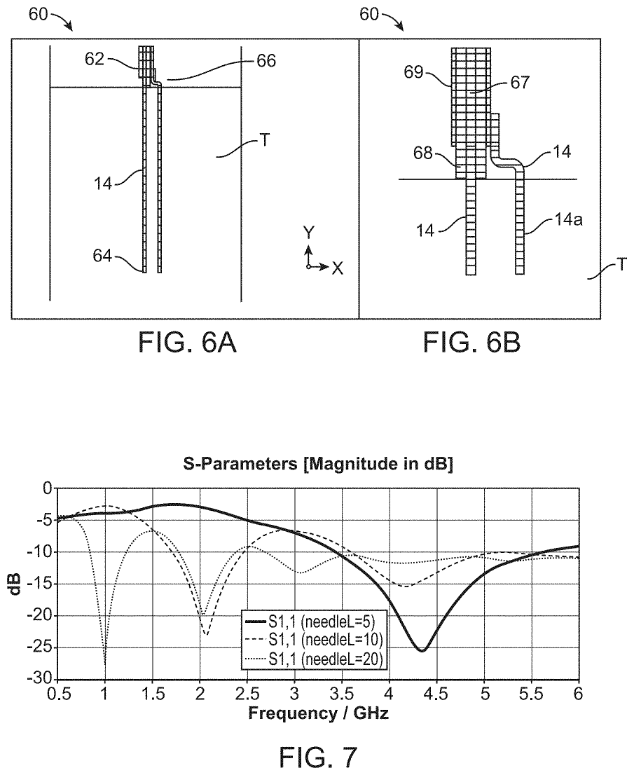

FIGS. 6A-6B show how a two-wire line 60 can be formed utilizing a coaxial cable 62. The center conductor 67 forms one of the arms of the needle 14, the other arm of the second needle 14a is formed by attaching a wire to the outer conductor (shield) 69 of the coaxial cable 62. The coaxial cable includes a shield 69 disposed over the center conductor 67 with a dielectric 68 disposed in between the two layers 67, 69. The two-wire line 60 has a region of first reflection 66 adjacent the distal end of the coaxial cable 62 and a second region of reflection 64 near the distal end of the needles 14.

FIG. 6A shows needles 14 extending into tissue T.

FIG. 6B illustrates an enlarged version of FIG. 6A.

When the above device and method seen in FIGS. 6A-6B is utilized, a first reflected signal may be present at the transition between the coaxial cable and the two-wire line at the first reflection region 66. This first reflected signal will interact with the second reflected signal at the open end of the two wires at the second region of reflection 64 to create frequency dependent variation in the impedance of the applicator device 60 (the two-wire line). The amount of the reflected signal at the coax to two-wire junction (first reflection 66) is dependent on the surrounding tissue type, the distance between the two wires, the diameter of the wires, and the frequency. The amount of reflected signal at the open end of the two-wire transmission line (second reflection 64) is dependent on the surrounding tissue type, the distance between the two wires, the diameter of the wires, the frequency and the length of the transmission line. For a desired frequency of operation and tissue type, the separation distance between the wires, the wire diameter and the length of the transmission line can be selected to optimize the amount of energy coupled into the applicator. Separation and diameter of wires will tend to affect depth of resonance. Length of transmission line will tend to vary the frequency of optimal coupling (resonant frequency). A first wire may be straight and interact with a dielectric layer 68 that is covered by a shield 69. The second wire may be coupled to the shield 69 and have a curved portion that separates the distance between the first needle 14 and the second needle 14a.

FIG. 7 shows the S11 (amount of reflected signal at the input coaxial cable) on a dB scale vs. frequency for a coaxial fed two wire transmission line embedded in muscle tissue. Three different needle lengths are shown: 5 mm (solid trace), 10 mm (dashed trace) and 20 mm (dotted trace). As shown in the figure, different optimal frequencies of operation (in which the reflected signal reaches a local minima) are demonstrated for the different lengths. For example, the 5 mm long needles have a single optimal operating point at approximately 4.4 GHz. The 10 mm long needles have two optimal operating points at a first resonance of 2 GHz and a second resonance of 4 GHz. The 20 mm long needles have three optimal operating points including a first resonance at 1 GHz, a second near 2 GHz and a third near 3 GHz. Each resonance will create a different standing wave pattern (and corresponding absorption pattern).

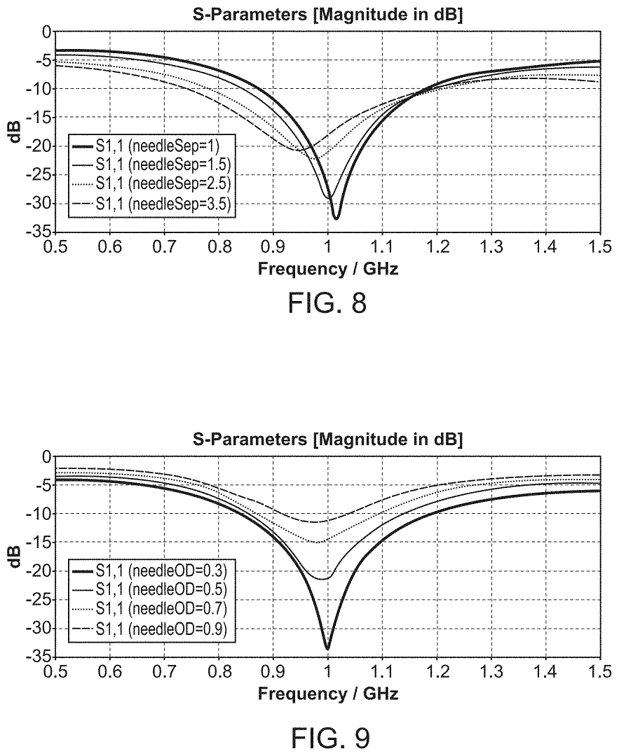

FIG. 8 shows the S11 (in dB) vs. frequency for 20 mm long needles embedded in muscle tissue with a fixed wire diameter (e.g. 0.3 mm) but varying separation distances. Changing the separation distance causes small shifts in the resonance while the depth of the resonance is affected more dramatically. Deeper resonance at the desired operating frequency translates to more efficient delivery of power into the device.

FIG. 9 shows the S11 (in dB) vs. frequency for 20 mm long needles embedded in muscle tissue with a fixed separation distance (e.g. 1.5 mm) but varying wire diameter. Changing the wire diameter causes small shifts in the resonance while the depth of the resonance is affected more dramatically. Deeper resonance at the desired operating frequency translates to more efficient delivery of power into the device.

For lower water content tissue types, such as lung or fat tissue, the dielectric constant of the surrounding tissue decreases. This has the effect of decreasing the electrical length of the transmission line and increasing the resonant frequency of the system.

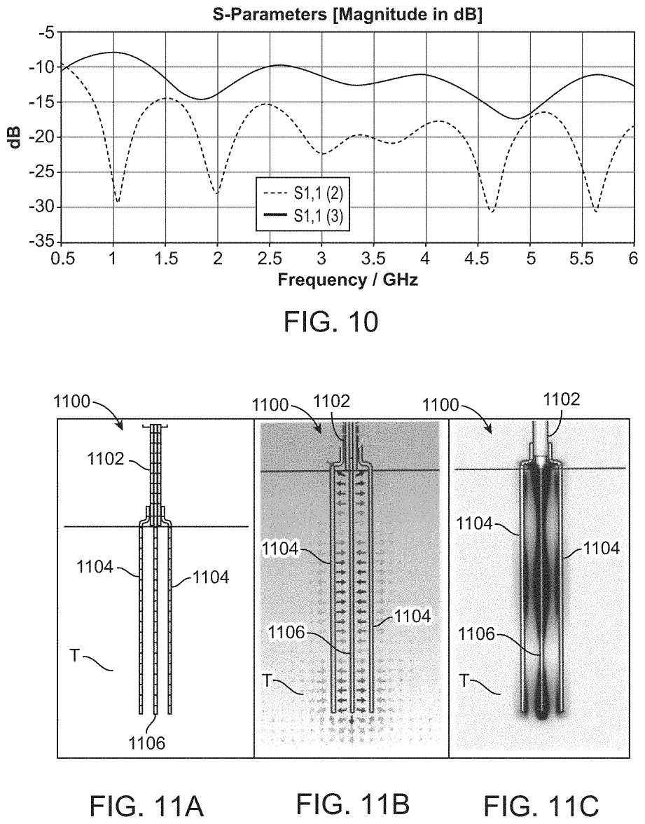

FIG. 10 shows the S11 vs. frequency of the coaxial fed two wire line previously shown in FIGS. 6A-6B, except embedded in lung tissue rather than muscle. The solid trace shows the S11 with the length of the transmission line set to 20 mm. Unlike in muscle tissue, where there is a first resonance at 1 GHz, the first resonance is closer to 2 GHz due to the inherently lower dielectric constant of lung tissue. If the length of the transmission line is increased to 35 mm (dashed trace) the first resonance at 1 GHz is restored and the second resonance is again at 2 GHz.

Using the concepts above, the transmission line length, separation distance, wire diameter and frequency can be utilized to create an microwave applicator device that can create adjustable lesion sizes in a variety of tissue types. In one example, the mechanical structure of the applicator can allow in-situ adjustment of the transmission-line length, with corresponding adjustment in frequency to allow optimal efficiency and targeting of variable sized tumors or cardiac tissue targets. In an alternate example, the applicator can have a fixed transmission-line length but be excited with a variable frequency to create an absorption pattern that targets variable sizes of tumor or cardiac tissue. In another example, the applicator can have a fixed transmission line length and the frequency can be changed for optimal efficiency and absorption pattern as the applicator comes into contact with different types of tissue (such as going from muscle tissue to fat or lung tissue). In another example, the applicator can have a variable length such that the length can be changed for optimal efficiency and absorption pattern as the applicator comes into contact with different types of tissue.

Example Ranges:

Any of the needles disclosed herein can be adjusted to operate in a range of tissue types including fat, muscle, tumorous tissue, lung, cardiac and other soft tissues. This spans a range of dielectric constants from 5 to 100.

Separation distances may be 0.5 to 15 mm between needles. Nominal range may be 1 to 10 mm.

Wire diameter of a needle may range from 0.1 to 2 mm. Nominal range may be 0.15 to 0.5 mm.

Transmission line lengths may range from 1 to 100 mm. Nominal range may be 2 to 30 mm.

Transmission line impedance range may be: 10 to 320 Ohms.

Frequency range may be: 400 MHz to 10 GHz. Lower frequencies lend themselves to larger absorption pattern, higher frequency is more localized to the region of the antenna.

Any of the examples of microwave delivery devices described herein may have any or all of the parameters described above in any combination or permutation.

Multi-Pronged Inner or Outer Conductor

A two-wire transmission line can be expanded to a multi-line transmission line 1100. This includes multiple negative or positive lines 1104, 1106.

FIGS. 11A-1C show a three-wire transmission line 1100, with 20 mm length lines 104, 1106, operating at 2 GHz. This example includes a three-wire transmission line 1100 with two outer needles 1104 and an inner needle 1106 coupled to a coaxial cable 1102. The outer needles 1104 may vary in length and diameter relative to one another or relative to the inner needle 1106.

FIG. 11A shows an example where the design has an inner conductor 1106 with an outer conductor 1104 on either side.

FIG. 11B shows the E-fields along the transmission line of FIG. 11A and illustrates are largely constrained to the region between the inner and outer conductors as indicated by the arrows. This is similar to the two-wire transmission lines previously discussed above.

FIG. 11C shows the absorption pattern (scaled to 1/10 of max) indicated by the darkened regions of the example in FIGS. 11A-11B.

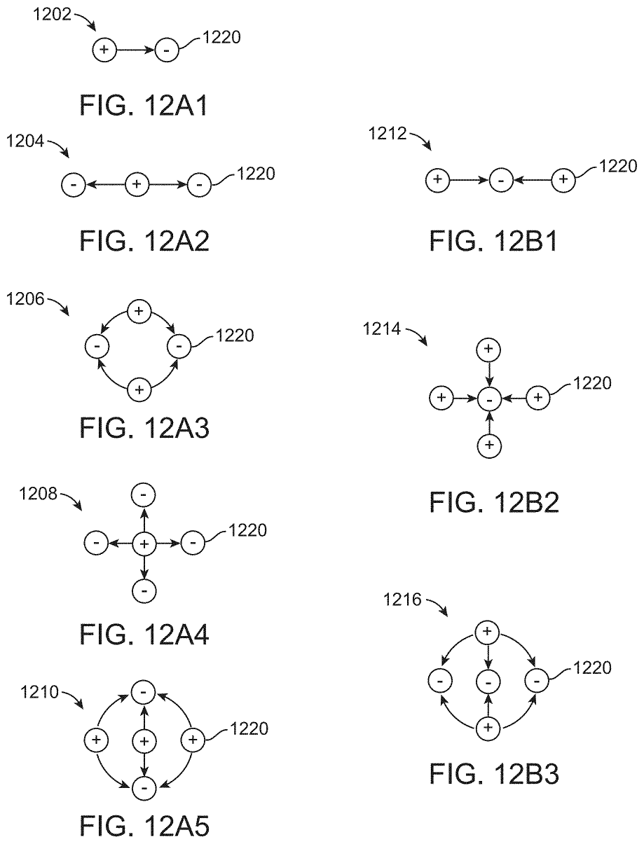

FIGS. 12A1-12A5 and 12B1-12B3 show a cross section through various configurations of wire transmission lines.

FIG. 12A1 shows a two-wire configuration 1202 with one positive and one negative wire 1220 in a linear array.

FIG. 12A2 shows a three-wire configuration 1204 with two negative and one positive wire 1220 in between the two negative wires, and in a linear array.

FIG. 12A3 shows four-wire configuration 1206 with two positive and two negative wires 1220 circumferentially disposed about ninety degrees apart from one another. The positive and negative wires alternative circumferentially.

FIG. 12A4 shows a five-wire configuration 1208 with one positive wire in a center position surrounded by four negative wires 1220 forming a cross-shaped pattern. The four negative wires are arranged approximately ninety degrees apart from one another circumferentially.

FIG. 12A5 shows another five-wire configuration 1210 with a central positive wire 1220 surrounded by four alternating positive and negative wires 1220 circumferentially disposed about ninety degrees apart from one another.

FIGS. 12B1-12B3 shows that polarity may be reversed in the three- and five-wire examples of FIGS. 12A2, 12A4, and 12A5.

In FIG. 12B1 a three-wire configuration 1212 with a negative polarity wire in between two positive polarity wires 1220 arranged in a linear array.

FIG. 12B2 shows a negative central wire 1220 surrounded by four positive polarity wires 1220 disposed about ninety degrees circumferentially apart from one another to form a five-wire configuration 1214.

FIGS. 12B3 shows another five-wire configuration 1216 with a negative central wire 1220 surrounded by four wires with alternating positive and negative polarity and disposed approximately 90 degrees circumferentially apart from one another.

Multiple conductors can be utilized to tailor the field configuration and corresponding absorption pattern of the transmission line applicator as shown in FIGS. 12A-12A5 and 12 B1-12B3. The center conductor may be attached to the positive terminals and the negative terminals may be attached to the outer terminals, or the opposite connections may be used.

Loop—Shorted Two-Wire (or Multi-Wire) Transmission Line

The transmission lines described above can be terminated in a short circuit, rather than an open circuit. In this case there still exists a first reflection at the coax-to-two-wire transition (near the distal end of the coax cable) and a second reflection at the short circuit termination near the distal end of the transmission lines. This second reflection due to a short circuit has a different phase than an open circuit and therefore the length of line that optimizes the efficiency at a particular frequency is different. Thus, the use of a shorted line can be utilized to create an absorption pattern of a desired length/size while maintaining an optimal efficiency.

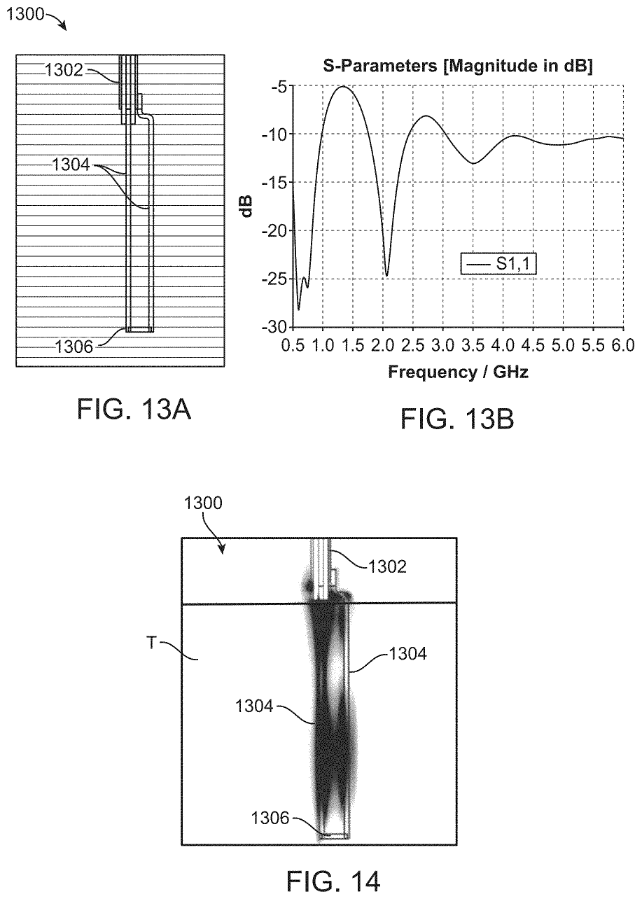

FIG. 13A shows a two-wire 1304 shorted transmission line 1300 design that is 15 mm long. The two wires 1304 may be needles for piercing tissue and one wire is coupled to the inner conductor of the coaxial cable 1302 while the other wire is coupled to the outer conductor of the coaxial cable. A connection 1306 creates a short between the two wires 1304 at or near their distal end.

FIG. 13B shows the S11 vs frequency for the design of FIG. 13A. The design has a second resonance near 2 GHz as previously shown for the open two-wire line, but in this case the length of the line required to achieve this resonance is 15 mm rather than 20 mm. The result is a more compact heating pattern, as shown in FIG. 14.

FIG. 14 shows the absorption pattern of a 15 mm long, shorted two-wire coaxial fed line 1300 in muscle tissue T. The darker regions between the wires show greater absorption compared to the lighter regions.

Loop—Standard Loop Antennas, Coupled to Magnetic Field

In another example, a loop antenna can be formed by forming a loop with e.g. the center conductor of a coaxial cable, bending it back toward the outer conductor and electrically attaching it to the outer conductor. The loop structure causes a large amount of current to flow in a circularly shaped (or partially circularly shaped) pattern, creating a magnetic field and subsequent electric field that gets absorbed in the surrounding tissue.

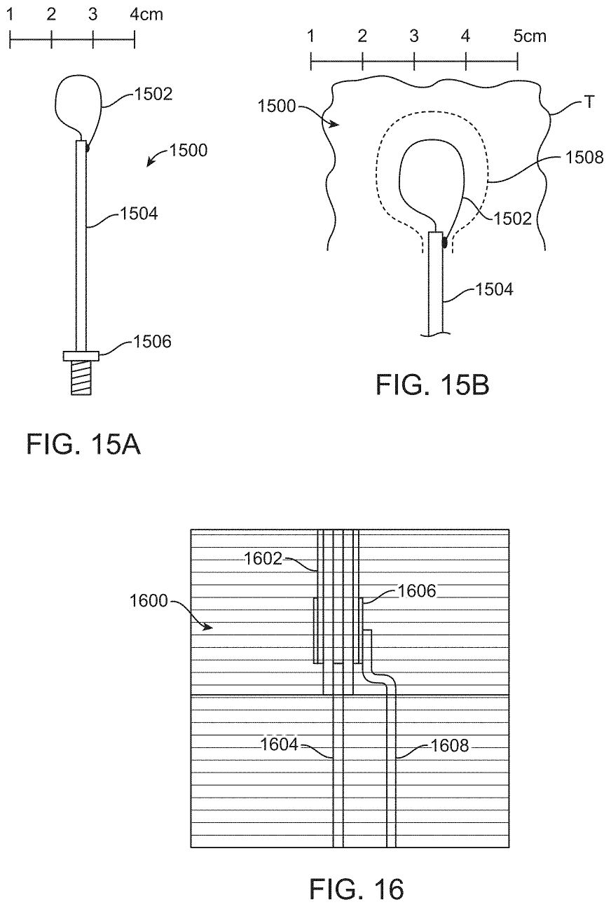

FIGS. 15A-15B show an example of a loop antenna 1500.

FIG. 15A shows construction of loop from a coaxial cable, here the loop 1502 may be formed from the center conductor of coaxial cable 1504 or any other conductor that is electrically coupled with the outer conductor of the coaxial cable 1504 to form a loop of any size or shape and disposed at the distal end of the coaxial cable. A cable connector or other connector element 1506 may be coupled to the proximal end of the coaxial cable to allow connection with a microwave generator or another instrument.

FIG. 15B show an example lesion 1508 created with the loop 1500 in FIG. 15A and disposed in tissue T (such as the liver).

The absorption zone of such a loop antenna is focused in the region where the loop is, as well as inside the loop. As such it may be adapted such that the loop encircles or partially encircles e.g. a tumor or other structure to preferentially target that structure and protect surrounding normal tissues.

Floating Outer Conductor/Floating Inner Conductor

Wires can be left electrically floating (from a low frequency standpoint), with microwave currents capacitively or inductively coupled onto the wires. This can be done for the center conductor and/or outer conductor of a coaxial cable.

FIG. 16 shows a coaxial fed two wire transmission line 1600, with the outer wire 1608 isolated from the shield of the coaxial cable 1602 with an insulator 1606. The inner wire 1604 may be directly connected to the inner conductor of the coaxial cable 1602. Microwave energy can be capacitively or inductively coupled onto the outer wire 1608 from the shield of the coax cable. The insulator 1606 may be created from plastic or ceramic materials.

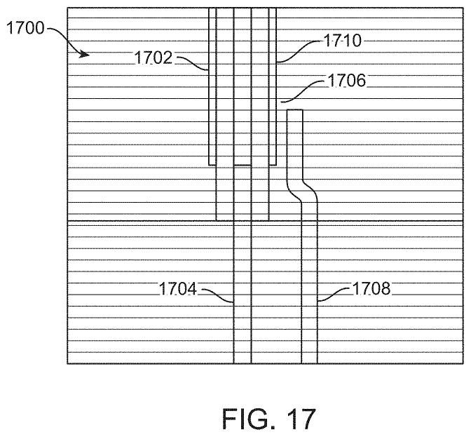

Additionally, rather than using a plastic or ceramic insulator, the outer wire can be embedded in tissue and placed near the shield of the coaxial cable. In this configuration microwave energy can couple onto the outer wire across the tissue. This is shown in FIG. 17 with a coaxial fed two wire transmission line 1700, where the outer needle 1708 is coupled to shield 1710 through tissue 1706. Other aspects of the transmission line 1700 are similar to those of FIG. 16 where an inner needle 1704 is coupled to the center conductor of coax cable 1702.

Leave-in-Place Conductor/Fusible Link

A fine flexible platinum or other metallic conductor can be looped around the target site. This can be accomplished number of ways. In an example, a nickel-titanium needle reshaped in the form a circle is advanced from a catheter. Once the penetrating needle encircles the target tissue and returns to the catheter tip, a conductor is fed through the need and recaptured. Microwave power is applied to the conductor causing the ablation of the target tissue. Once the ablation is successfully completed, a fusible link connecting the conductor to the catheter is melted by applying high current.

Differential Length Conductors

FIG. 18 shows a two-wire transmission line 1800 with unequal length lines 1802, 1804. The lengths of the wires for the two-wire transmission line can be set to unequal lengths. Having unequal length creates variation in the open-circuit effect at the end of the line. The variation can serve to change the magnitude or phase of the second reflection previously described above, potentially leading to a different optimal length for the transmission line. Additionally, fringing fields that naturally exist at the open ends of the transmission line may be varied to change the size/volumetric geometry of the absorption pattern in tissue. Here, the center needle or line 1802 is coupled to the center conductor of the coaxial cable 1806 and the outer needle or conductor 1804 is coupled to the shielding of the coaxial cable. The outer needle 1804 is shorter than the inner needle 1802. In another example, the inner needle 1802 may be shorter than the outer needle 1804.

Loop with Floating Loop

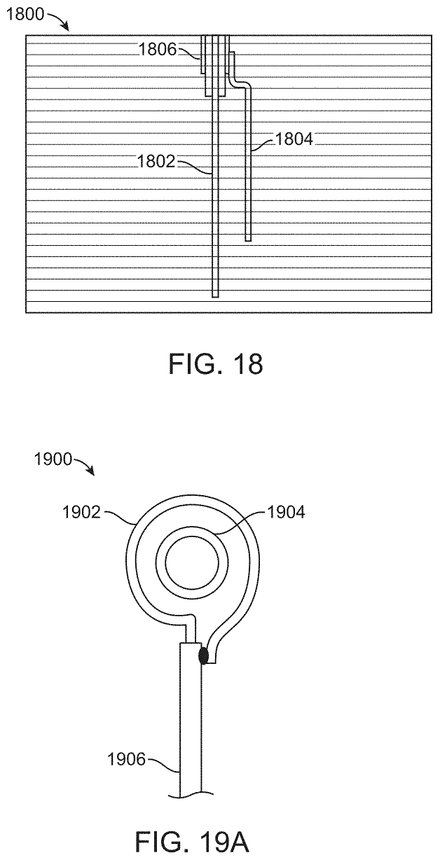

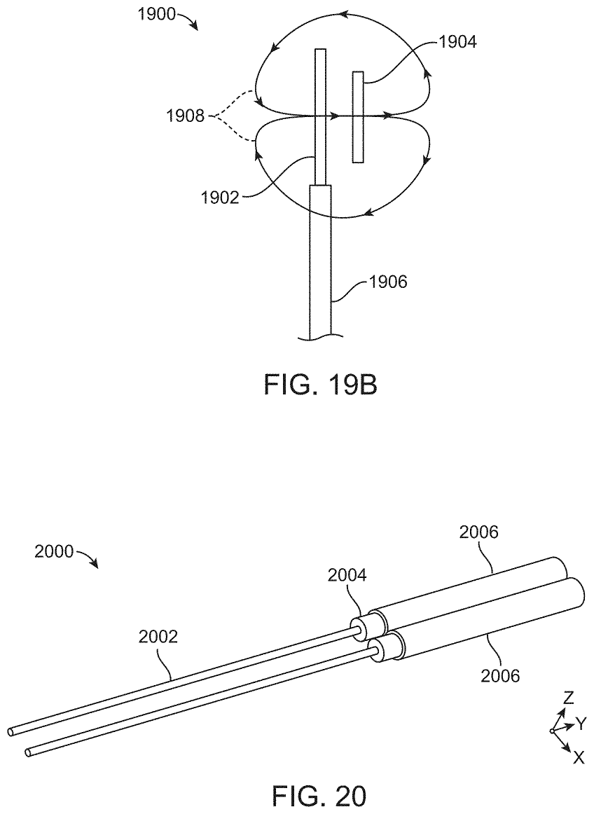

FIGS. 19A-19B show a loop antenna 1900 with a first loop 1902 and a secondary floating antenna 1904. FIG. 19A shows a front view of the example and FIG. 19B shows a side view of FIG. 19A.

In FIG. 19A, and outer loop 1902 may be formed with a wire or other conductive filament coupled to the center conductor of a coaxial cable 1906 at one end of the loop and the opposite end of the loop coupled to the outer conductor of the coaxial cable 1906. The loop may be any shape or size such as a round or circular loop. The inner loop or floating loop 1904 may also be a round loop or any other size and shape and may be disposed entirely or partially within the outer loop and in the same plane or in a separate plane.

FIG. 19B shows an example where the outer loop in in a first plane and the floating loop in in a second plane that may be parallel with the first plane. The resulting magnetic field 1908 between the outer loop and floating loop forms to loops which flow in opposite directions. For example, the two loops may be semi-circular in shape with one loop on top of the other loop. The top magnetic field runs counterclockwise while the bottom magnetic field runs clockwise. The two magnetic field share a common section of their perimeter.

The loop antenna geometry described previously in FIGS. 15A-15B creates currents traveling along the wire in a circular path, and the corresponding magnetic field created by these currents may be coupled into a floating loop of wire present in the tissue as shown in FIGS. 19A-19B. This magnetic coupling mechanism can result in induced currents in the floating loop, with resulting heating occurring in the region of both the main loop and the floating loop. This mechanism may be utilized for increasing the heating zone of a loop antenna, as well as implementing a leave in place conductor for identification of tumor margin.

Multi-Frequency Generator

A multi-frequency generator can be utilized for exciting any of the applicator devices described herein with a variable frequency signal for creating adjustable sized lesions. The generator may have a switchable frequency (e.g. in which one frequency can be output at a time) or may have the ability to generate a multi-frequency signal (e.g. in which multiple frequencies can be output at a time). A multi-frequency generator could be readily implemented with a combination of programmable or fixed synthesizers/oscillators, amplifiers, diplexers/multiplexers, combiners and filtering.

In addition to exciting different frequencies, the generator may be designed to measure reflected power at one or more frequencies. By measuring the magnitude and/or phase of the reflected signal and comparing it to the forward power signal, diagnostic information about the current length of the applicator transmission line and/or the type of tissue in contact with the tissue may be obtained.

Dual Coaxial (In/Out Phase)

FIG. 20 shows a microwave applicator device 2000 with dual coaxial cables 2006 (also referred to herein as coax) and dual conductor needles 2002 in a perspective view.

An alternate to the single coax fed two wire line discussed previously is a device 2000 with dual coax 2006. Here each needle 2002 is coupled to the central conductor of the coax cable. A dielectric 2004 is disposed over the central conductor. The dual coax configuration allows for a more balanced design than the single coax that uses a wire attached to the shield of the coax. In the balanced design the two wires (or needles) 2002 are fed 180 degrees out of phase such that one wire is positive while the other is negative. This achieves an absorption zone in between the two needles that is similar to the previous two-wire configurations shown above, with better symmetry between the needles possible.

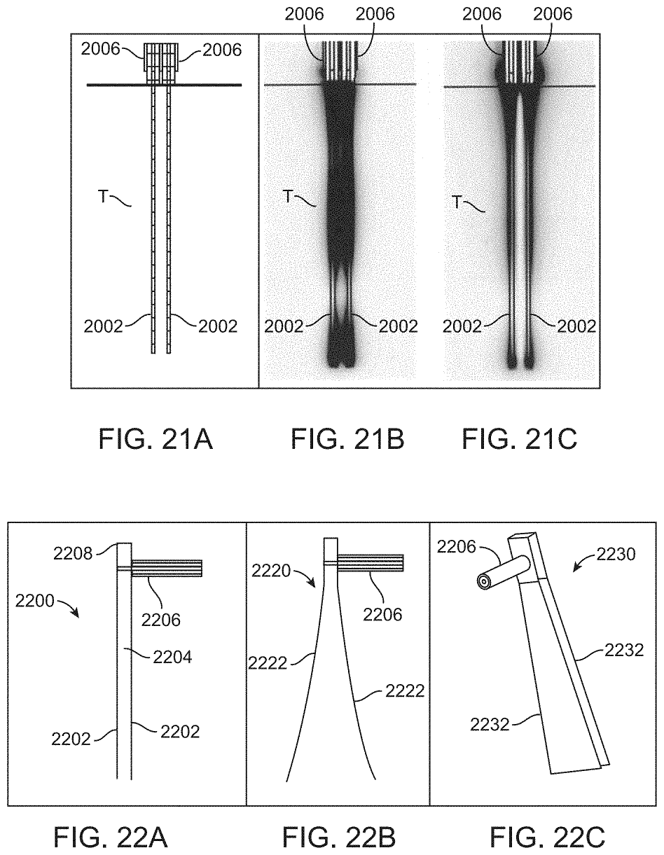

FIGS. 21A-21C show absorption patterns at 2 GHz for a dual-coax needle configuration such as the example in FIG. 14. FIG. 21A shows a cross section of the dual coaxial cables 2006 coupled to dual needles 2002 which are disposed in tissue T.

FIG. 21B shows 180 degree out of phase drive of the coaxial cables 2006, with a peak absorption zone is focused in between the two needles 2002 (black/dark areas indicate peak absorption). Lighter areas indicate less absorption.

FIG. 21C shows in phase drive of the coaxial cables 2006—there is a null absorption zone in between the two needles 2002.

In an alternate example shown in FIG. 21C, the two needles 2002 are coupled to two coaxial cables 2006 and may be setup to create two individual absorption zones by driving both coaxial needles 2002 in-phase. This results in a null absorption zone in-between the two needles 2002.

Phase Changing Microwave Generator

A microwave generator may be setup to drive two or more coaxial lines with a varying phase relationship. Thus, allowing an in-phase or 180 degrees out of phase type of absorption to be created as described above. The generator could also be utilized to drive the coaxial lines using a zero to 360-degree variable phase shift between coaxial lines. This could readily be done using a digital phase shifter, varactor or mechanical phase shifter.

Active Heat Management of the Coaxial Cable

In microwave technology, a medical device used to ablate tissue in the body cavity or in the vascular system often requires a long coaxial cable. Even when impedance is matched properly, a substantial amount of energy is lost in transmission through the cable. This causes heating of the cable. For treatments of a long duration, the coaxial cable could get hot causing unintended consequences. It may be desirable to provide a heat exchanger or cooling mechanism to alleviate excessive heating. In one example, a tight flexible sleeve is attached to the outside of the coaxial cable. The sleeve is fused/glued to the ends of the coaxial cable in such a way that a cooling element such as a fluid like a solvent can be introduced in the annular space between the outer shield and the sheath, and be fully sealed and contained within that space. The proximal end of the cable is cooled with cryogen or coolant. Material such as industrial diamond can be used to improve the efficiency of the cooling. During the application of the microwave energy, the heat generated in the coax cable will cause the liquid trapped to heat up and evaporate. The transfer of the energy will cool down the cable. The heated solvent or the vapor will reach the cooled proximal section of the device and condense back to liquid. The coaxial cable with the trapped solvent acts as a heat pipe transferring heat from the cable.

Flared Conductors

In addition to the two-wire or multi-wire transmission lines shown above, which are parallel and thus have a uniform impedance. A design with flared wires is also possible. A design with flared wires will have a non-uniform impedance, as well as a non-uniform electric field vs. distance down the line. The non-uniformity may be utilized to alter the impedance/efficiency and absorption pattern along the transmission line.

The flare angle can vary anywhere from 0 to 60 degrees. The nominal flare angle would be 0 to 30 degrees to maintain the transmission line effect as the signal propagates down the line.

Conductive Sheets, Conductors on PCB

As an alternative to wires, needles or other elements, the transmission lines presented above may be created with metal or otherwise conductive sheets of material. The sheets can be fed with a coaxial cable coupled in a perpendicular or transverse direction to the sheets of material, or in parallel alignment. In a perpendicular feeding configuration, the upper region of the transmission line may be open or short circuited, with the length from the feed to the open or short circuit being adjusted for coupling efficiency between the coax and transmission line. This feeding configuration may also be used for the wire-type of transmission line. The sheets may have uniform width or may have a step or taper as shown in the figures. Additionally, the separation distance between the sheets may also be tapered.

FIGS. 22A-22C show an applicator device design created using metal sheets rather than wires.

FIG. 22A shows a side view of applicator 2200 which also includes a perpendicularly aligned coax cable 2206 coupled to one end of the sheets 2202 which may be flat planar sheets of conducting material that form transmission line 2204. The applicator also may include an adjustable opening or short 2208 for tuning.

FIG. 22B shows an alternate version of applicator device 2220 with flared sheets 2222 to create non-uniform separation distance. The coaxial cable 2206 may be coupled perpendicular or transfer to the flared sheets, or parallel thereto.

FIG. 22C shows a perspective view of another alternate version of applicator 2230 with sheets 2232 of non-uniform width. The coaxial cable 2206 may be coupled parallel, perpendicular or transverse to the sheets 2232. FIG. 22C shows a perpendicular connection.

The examples of FIGS. 22A-22C can be designed to operate when embedded or pressed into tissue. However, they can also be designed to operate in a balloon or other media in which there aren't significantly lossy materials in-between the two arms of the transmission line. In this fashion, an end-fire type device that mainly creates absorption at the distal end of the device, may be created.

An alternate example can include traces on a substrate such as a Printed Circuit Board (PCB) rather than wires to form a two-wire, multi-wire or loop type of applicator. The PCB may be attached to a coaxial cable, which feeds into traces on the PCB rather than wires. This may be utilized for creating a particular mechanical structure, for example a more rigid structure for puncturing a tumor or other desired target tissue. The PCB can be conventional circuit board such as FR4, high frequency circuit board such as Rogers or Taconic laminates, or may also be built from ceramic materials such as Macor or Alumina. Examples are shown in the figures.

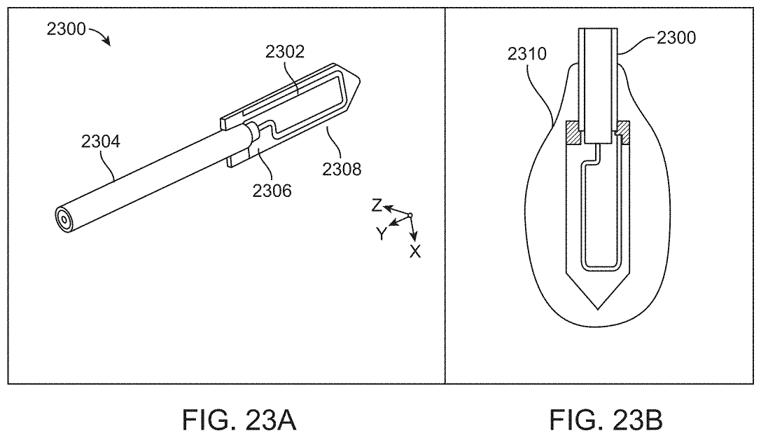

FIGS. 23A-23B show a PCB based loop applicator design 2300.

FIG. 23A shows a perspective view of the applicator 2300 showing the coaxial cable 2304 to PCB 2308 transition 2306 and the loop 2302 implemented as traces on the PCB 2308. Optionally one end of the trace may be coupled to the center conductor of the coaxial cable and the other end of the trace may be coupled to the shielding of the coaxial cable.

FIG. 23B shows an example of a heating/lesion pattern 2310 of PCB loop applicator 2300 previously illustrated in FIG. 23A.

Temperature Controlled Microwave Ablation

With proper filtering, thermocouples or thermistors can be used with the microwave devices disclosed herein to measure temperature of tissue during ablation. This configuration can work in most cases, but in some special cases an optical temperature sensing method can be used with the examples disclosed herein instead of a thermocouple or thermistor. In one example, hollow conductors are used to form the electrode elements of any of the above-mentioned configurations. Further, a fiber optic temperature probe (such as Luxtron) is placed in the inside of the hollow conductors. This allows the continuous monitoring of the ablation temperature without distorting the microwave field.

A smart microwave generator may be used where temperature-controlled ablation can be accomplished using the temperature reading from the fiber optic probes or any other temperature sensor used (e.g. thermistor, thermocouple). The generator can be programmed to vary the power output to maintain a preset temperature target.

Method of Inserting Electrodes into the Tissue

The electrode of any example herein can be inserted into the target tissue by mechanical means or by the means of an electromechanical feature integrated into the device. A tissue piercing tip may be included in any of the examples to facilitate introduction into the tissue.

The coaxial cable used for the transmission can be chosen such that it has sufficient pushability and control to force electrodes with sharpened tips to enter the tissue. The outer shaft of the device can be made steerable as to allow the electrodes to pass through the target tissue in the correct orientation. This would allow the user to ablate any desire aspect of the tissue.

The electromechanical insertion of the electrode needle(s) can be achieved by incorporation solenoid like feature at an appropriate location near the distal end of the catheter. When actuated, the inner core with the electrode is advanced. The rate of advancement can be controlled by the amount of current applied to the coil.

Penetrating a tumor mass in soft tissue such as lung or liver with large probes is often difficult as the tumor mass tends to move away. In any example, a central thin sharp needle with barbs may be first advanced from the tip of a catheter to penetrate and stabilize the tumor or other target tissue. This central needle may be a passive element, or it may be a conductor for delivery of microwave energy into the tissue. Subsequently, the additional conductors are advanced into the stabilized tumor for ablation.

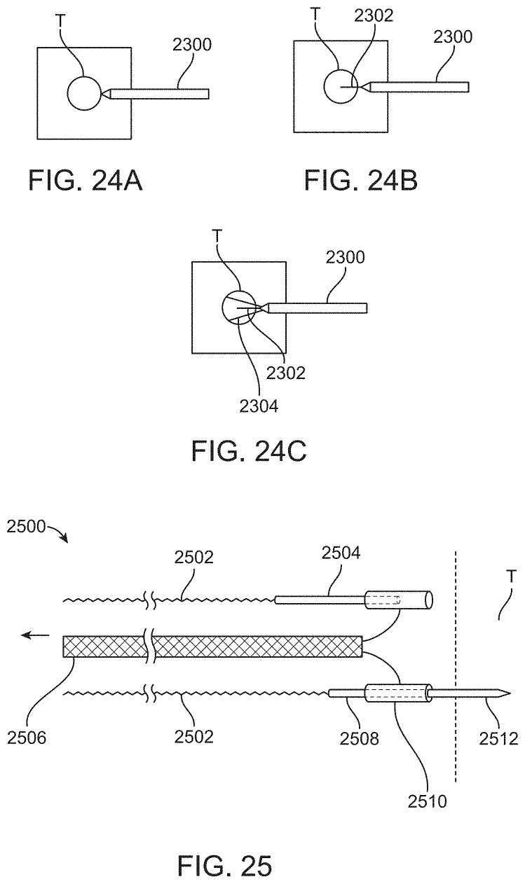

FIGS. 24A-24C show an ablation catheter 2300 penetrating the target ablation tissue T. The ablation catheter has an anchor element 2302 which allows the ablation catheter to be stabilized in the tumor. In an example, there may be one anchor element to stabilize the target tissue. In another example, more than one anchor element may penetrate the target ablation region and stabilize the tumor. In any example, the one or more anchor elements 2302 penetrate the target ablation zone region before the one or more transmission lines 2302 penetrate the target ablation region. In another example, the transmission lines penetrate the target ablation region before the one or more anchor elements penetrate the target ablation region. In another example, the transmission lines penetrate the target ablation region concurrently with the one or more anchor elements. FIG. 23A shows the catheter advanced adjacent to the treatment region T. FIG. 23B shows advancement of the anchor element 2302 distally into the target treatment region, and FIG. 23C shows subsequent deployment of the transmission lines 2304 into the target treatment area. Anchoring elements may be included with any of the devices or methods disclosed herein.

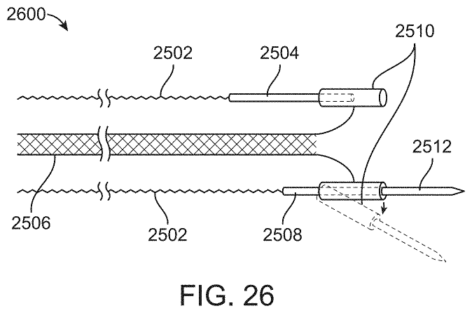

FIGS. 25 and 26 show a schematic view of retractable needle electrode catheters 2500 and 2600. At the distal tip of the device, the coax cable 2506 is connected to two tubular needle electrode guides or contacts 2510. A needle electrode 2504, 2508 is connected to a non-conductive shaft 2502 is inserted through the needle guide 2510. The inner diameter of the needle guide has a spring loaded contact to maintain good electrical connection between the needle guide 2510 and the needle 2504, 2508. Further, the needle guides may be designed to pivot in plane (independently or in unison) to change of angle of entry of the needle electrode into the tissue T. At the proximal end the needle actuator shafts 2502 are connected to an actuator mechanism in the handle (not shown). The needle electrodes can be actuated together or independently of one another such that one needle 2512 may be disposed in the target tissue T while the other needle 2504 remains in a retracted configuration.

In FIG. 25 the needle electrode 2504, 2508 may be actuated individually or together in a proximal or distal direction that is substantially parallel with the longitudinal axis of the device. The needle electrode actuator shafts may be actuated manually by an operator or they may be coupled to an actuation mechanism such as a motor, piston or other mechanism for moving the actuator shafts. One electrode needle may be coupled to the shielding of the coaxial cable 2506 and the other electrode needle may be coupled to the center conductor of the coaxial cable 2506, or the electrode needles may be coupled in any other configuration such as those described herein. More than two electrode needles may be employed in this device. FIG. 25 shows one needle deployed while the second needle is still disposed in the electrode guide.

FIG. 26 shows a variation of the example in FIG. 25 with the major difference being that the electrode needles may be pivoted relative to one another to change the angle of insertion. This may be done manually or an actuation mechanism such as a motor or other pivoting mechanism may be used. Other aspects of the example in FIG. 26 are generally the same as those in FIG. 25.

Example Devices for Ablation

An example of a design for tissue (e.g. cardiac, lung, tumor, or other tissue) ablation applications has a four wire transmission line with two positive conductors attached to the inner conductor of a coaxial cable and two negative conductors connected to the outer conductor of a coaxial cable as shown below. The coaxial cable has an outer diameter of 0.047″ (a standard “047” coaxial cable).

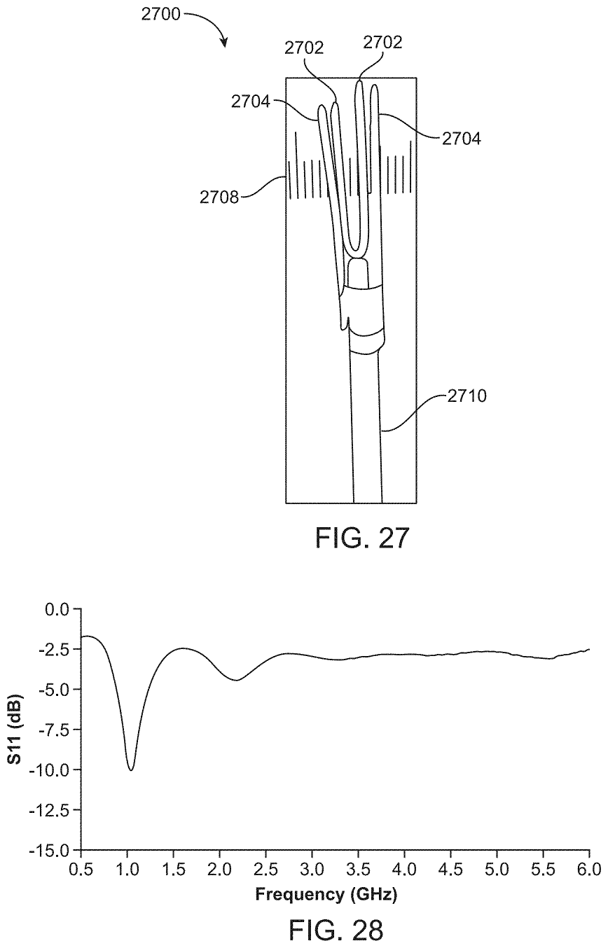

FIG. 27 shows an example ablation device 2700 which includes two positive needles 2702 and two negative needles 2704 offset roughly ninety degrees apart from one another in a circumferential direction. The needles 2702, 2704 are coupled to a coaxial cable 2710. A scale 2708 in millimeters show an example size of the working tip of the device. The device may be designed for cardiac or other tissue ablation with the following non-limiting specifications:

Separation distance: 5 mm between needles.

Needle wire diameter: 0.0287 inches (0.729 mm).

Transmission line length: 20 mm.

Transmission line impedance: 20 to 100 Ohms.

Operational frequency: 900 to 1100 MHz, operates at the first resonance (second or third is also possible).

FIG. 28 shows S11 vs. frequency of a nominal design for cardiac ablation. First resonance shown near 1 GHz, second resonance shown near 2 GHz.

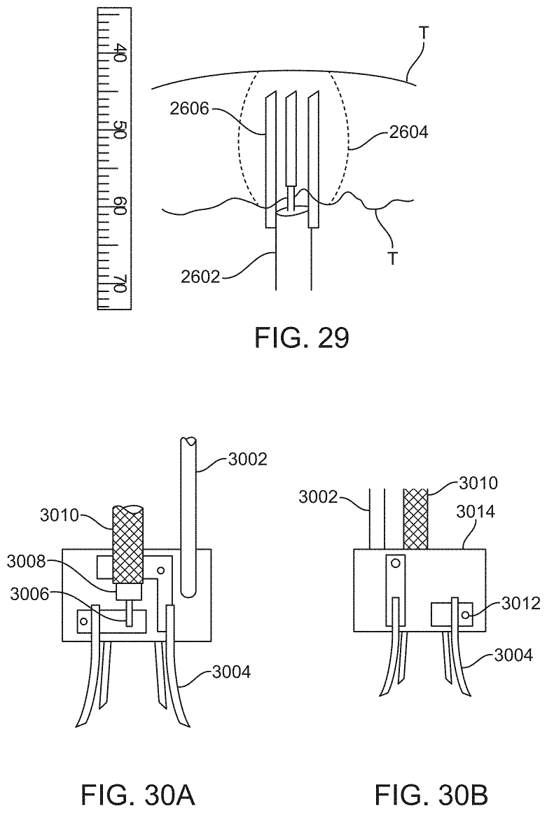

FIG. 29 shows an example of a transmural lesion 2604 created in tissue T such as cardiac ventricular tissue, lung tissue, or any other tissue, with an example device which includes three needles 2606 coupled to a coaxial cable 2602. In this example the two outer needles may be coupled to the shielding of the coaxial cable and the middle needle may be coupled to the center conductor of the coaxial cable. The scale in this figure is shown in centimeters to demonstrate relative size of the device but it not intended to be limiting. Any figure in this specification which may have a scale to show relative size of the example is not intended to be limiting.

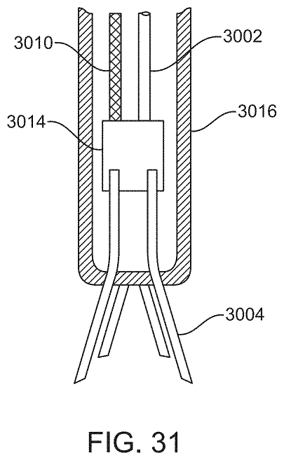

FIGS. 30A (front view)-30B (back view) and FIG. 31 show an example of a deployable needle device having a four-needle 3004 microwave probe. A coaxial cable's outer braid 3010 is soldered to a trace, separating it from the core 3006 wire with a dielectric 3008. The core wire 3006 is soldered to a separate trace. On the front face of the PC board 3014, are soldered 2 needles 3004, isolated from one another, but connected electrically to either the core wire 3006 or the coax braid 3010. The other two needles 3004 are coupled to the other of the core wire or coax braid. Also attached to the PC board is a plunger mandrel 3002 that will control the sliding of the needles in and out of a catheter tip 3016 (best seen in FIG. 31) and into the tissue. The mandrel is a solid rod and the coax cable follows the position of the mandrel.

On the back of the PC board, vias 3012 connect the two electrical pathways to traces on the back of the PC board and corresponding needles are soldered into place.

The needles 3004 may be hypodermic needles or sharpened mandrels. The needles can be coated with a lubricant, such as parylene to facilitate tissue penetration.

For electrophysiology applications, an ECG electrode can be threaded through a separate lumen and an ECG electrode placed at the distal tip of the electrode. So can a thermocouple or thermistor. A temperature sensor such as an optical fiber can be threaded through the central lumen of a hypodermic needle to measure temperature. The catheter tip has angled exit holes for the needles to launch at angles spreading their distances between each other in the tissue, or the needles may be pre-set in angle and take on their shape when exiting the catheter tip.

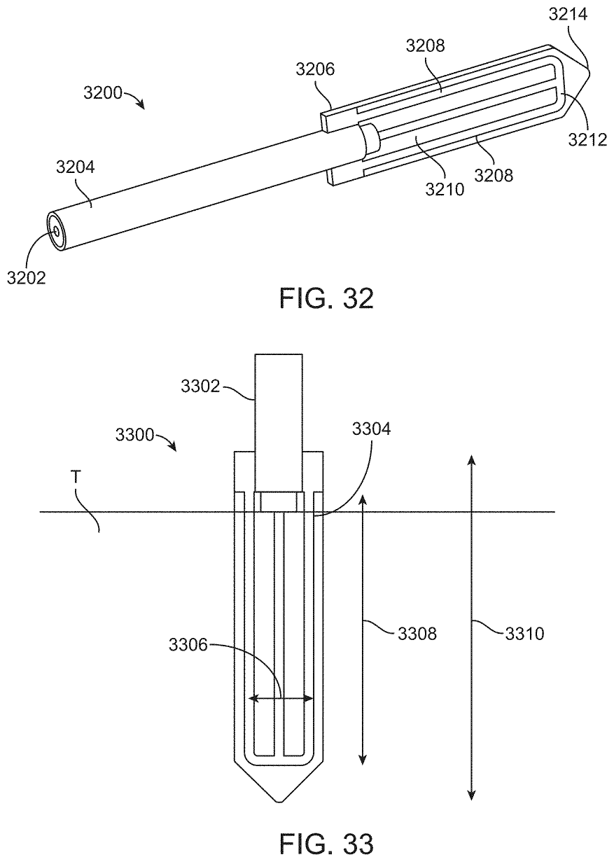

FIG. 32 shows a perspective view of nominal tumor ablation device 3200. The example has a three-wire transmission-line, with two outer conductors 3208 and one center conductor 3210. The center conductor may be coupled with the core of the coaxial cable and the two outer conductors may be coupled with the shield in the coaxial cable. The line is terminated in a short circuit 3212. It is constructed on a substrate 3206 such as a Printed Circuit Board (PCB) or ceramic and formed into a tip 3214 capable of penetrating tissues such as normal or tumorous lung tissue. The PCB is fed with 0.047″ sized coaxial cable 3204 that is flexible. The transmission-line is printed on both sides of the PCB such that the line is in close contact with tissue on both sides. The traces may be coated with an insulating layer such as parylene to help with the removal of the device from coagulated tissue. An example nominal configuration is shown below.

FIG. 33 shows a front view of an example of a tumor ablation device 3300 with non-limiting, example dimensions disposed in tissue T. The nominal frequency of operation for the device is 5.8 GHz, which is the second resonance for a line length of 6.8 mm. The impedance of the transmission line for the optimal design is 32 ohms (it can range between 15 and 45 ohms). Here the device includes three transmission lines with two outer and one inner trace disposed a substrate such as a PCB. The trace width may be about 0.25 mm. A coaxial cable 3302 is coupled to the transmission line and the inner conductor of the coaxial cable may be coupled to the inner conductor and the two outer conductors may be coupled to the outer shield conductor of the coaxial cable, or the connections may be reversed. The PCB may have a pointed tip to facilitate with tissue piercing. The loop width 3306 may be about 1.75 mm, the line length 3308 about 6.8 mm and the PCB length 3310 about 8.7 mm. These are example dimensions are not intended to be limiting.

FIG. 34 shows S11 vs. frequency of the tumor ablation design in FIG. 33, showing second resonance near 5.8 GHz.

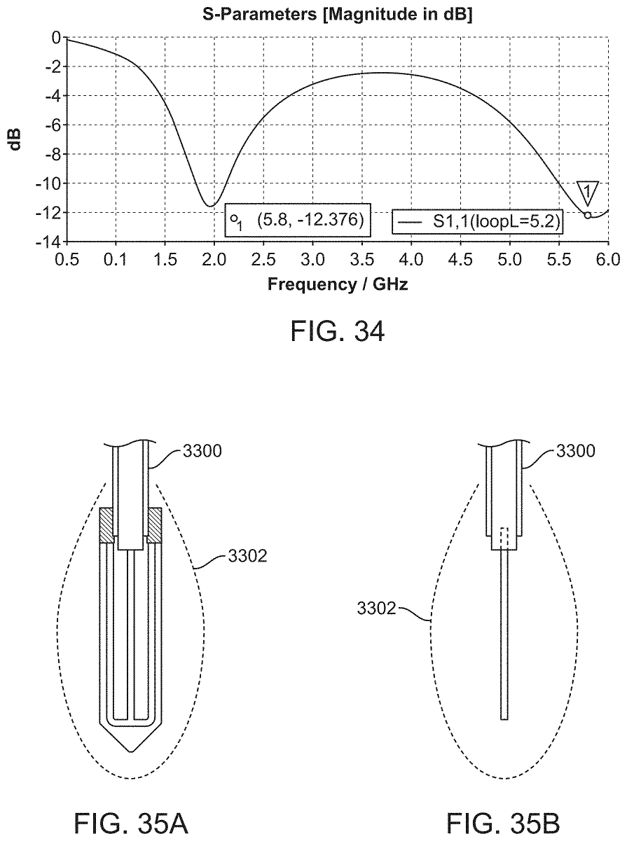

FIGS. 35A-35B show an example of the heating/lesion profiles from the tumor ablation device 3300 in FIGS. 32-33, operating at a frequency=5.8 GHz.

FIG. 35A shows a front view of the heating profile 3302 forming a partially elliptical profile.

FIG. 35B shows a side view of the heating profile 3302.

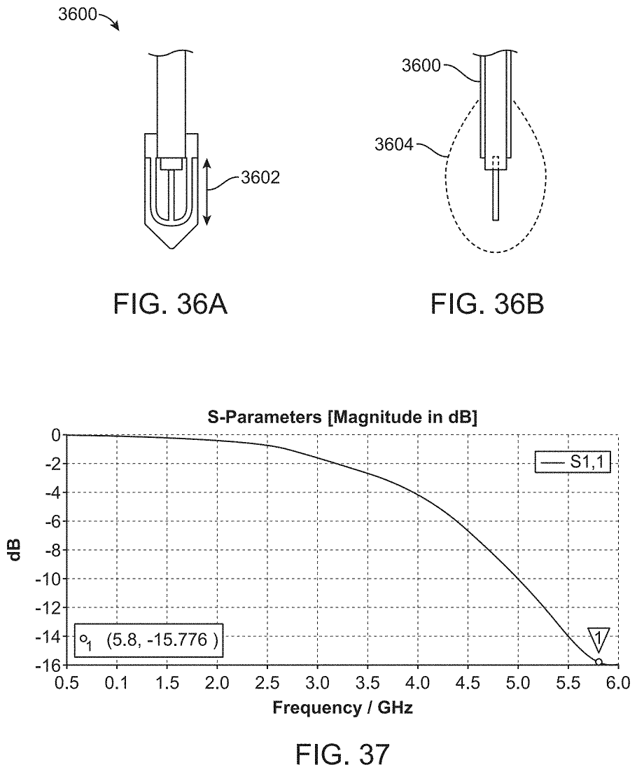

In variation of the example in FIGS. 32-33, the line length may be decreased to 2.8 mm such that the device operates at the first resonance for a frequency of 5.8 GHz, as shown FIGS. 36A-36B.

FIG. 36A shows the device 3600 with non-limiting example line length of 2.8 mm, and frequency=5.8 GHz. The device includes three conductors, two outer and one inner conductor that are coupled with a coaxial cable. The inner conductor may be coupled to the inner conductor of the coaxial cable and the two outer conductors may be coupled to the shield of the coaxial cable, or the connections may be reversed. The three conductors may be mounted on a substrate such as a PCB with a pointed tip to facilitate tissue penetration. Other aspects of the device 3600 are generally the same as the device in FIGS. 32-33.

FIG. 36B shows aside view of the heating/lesion pattern 3604 for the device 3600 of FIG. 36A. Here, the heating pattern is partially elliptically shaped.

FIG. 37 shows S11 vs. frequency of the 2.8 mm design in FIG. 36A. The first resonance is near 5.8 GHz.

Tuning for Optimal Energy Transfer

Tissue properties change as microwave energy is applied. Often this results in a mismatch of electromagnetic wave penetration into the body and high reflected power. Not only does the efficiency of the power transfer to the tissue go down but, the reflected power manifests as excessive heating of the coaxial cable. A hot coaxial cable can cause unintended burns in adjacent tissue or unwanted equipment damage.

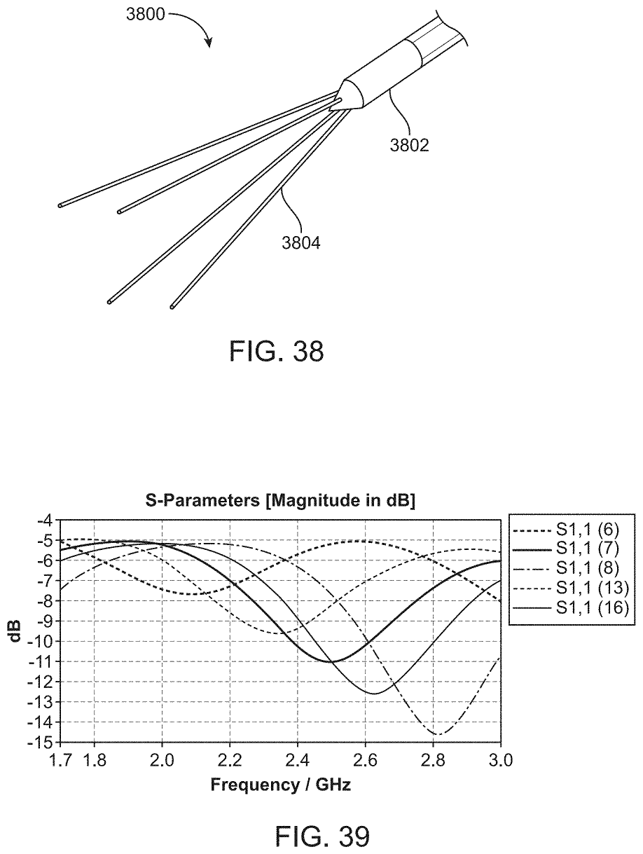

In one example, the conductor lengths are optimized deliberately for changing (denaturing, heating and losing moisture) desiccated target tissue as function of microwave application. Though there may be a mismatch for the native tissue, as soon as the ablation starts causing desiccation around the conductor, the match improves, and highly efficient transfer of the energy ensues for the rest of the ablation time. For a given frequency, the ideal conductor lengths for ablating desiccated tissue can be theoretically or empirically calculated. Conductors designed in this fashion are more efficient and produce minimal heating of the coaxial (also referred to herein as coax) cable. In one specific example, a design with 24 mm long conductors and a 25-degree flare angle is designed to ablate high water content tissue (such as liver, cardiac or muscle tissue) as shown in FIG. 38. This configuration was designed for operation at 2.45 GHz and the device 3800 includes four needles 3804 coupled to a coaxial cable 3802. In this example the needles flare radially outward and away from the coaxial cable.

FIG. 39 shows an example S11 vs. frequency plot for the device 3800 in FIG. 38, with the dielectric properties of the tissue reduced from the nominal value (corresponding to unablated tissue at regular human body temperature) to a value corresponding with ablated tissue. The figure shows that the resonance of the applicator is shifting upward as the dielectric properties change during an ablation. As a result of this shifting, the value of S11 at the operational frequency of 2.45 GHz varies.

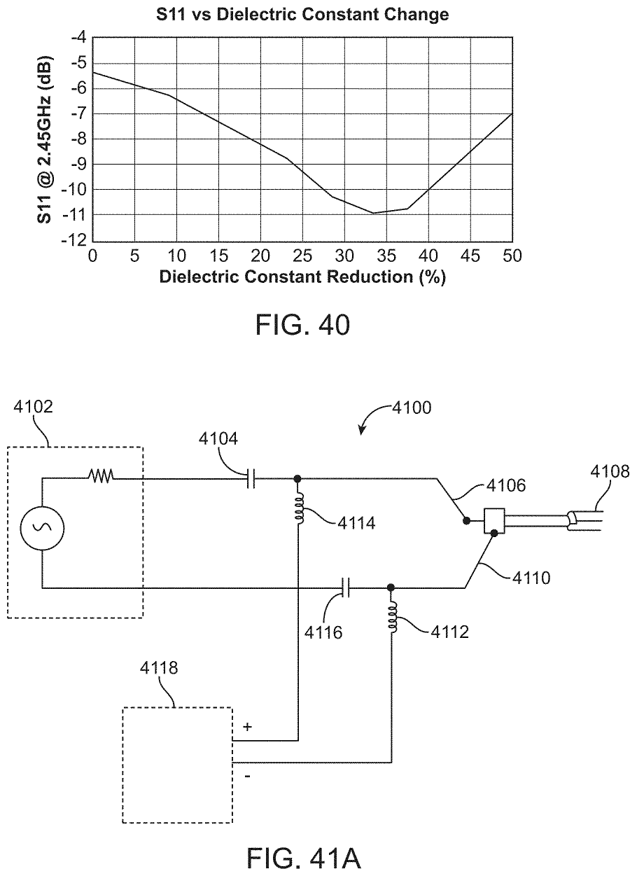

FIG. 40 shows that the S11 dB is minimized (and thus efficiency maximized) when the dielectric constant is reduced by around 35%. As the ablation progresses, the S11 starts to increase again (with efficiency dropping). This phenomenon may be utilized as a means of monitoring the progress of an ablation.

Injection of Solution to Target Site

In microwave ablation of tissue, good matching or tuning of the antenna is essential for efficient transfer of energy to the target tissue. Interestingly, the biological tissue properties vary widely, and a single antenna operating at chosen frequency may not be ideal for all tissue types. Table 1 below shows the representative values for heart, liver and lung tissues at an operating frequency of 2.4 GHz. In the current examples disclosed herein, an antenna made of hypotubes capable of delivering fluid to the tissue tuned to work most efficiently in the presence of a biocompatible solution is used to perform ablation of a variety of tissues. The biocompatible solution can be chosen from a variety of material known to medical professionals such as normal saline, phosphate buffered saline, dextrose solution (e.g. D5W), Lactate Ringer's solution, etc. The antenna can be designed for any chosen operating frequency. In an example, the antenna is tuned to work at one of the following frequencies: 915 MHz, 2.4 GHz or 5.8 GHz.

TABLE 1

| | | Elec. |
|---|---|---|
| | | Cond. |
| Tissue | Permittivity | (S/m) |
| Heart Muscle | 5.49E+1 | 2.22E+0 |
| Liver | 4.31E+1 | 1.65E+0 |
| Lung (inflated) | 2.05E+1 | 7.90E−1 |

In any example, the target tissue (e.g. tumor, infarcted heart tissue, aberrant or otherwise diseased or damaged tissue) is identified using appropriate diagnostic imaging methods such as CT scan, MRI, electro-anatomical mapping, ultrasound, etc. Using a fine hypodermic needle, a chosen solution (e.g. saline or D5W) is injected to cover the entire target tissue. The solution may also contain other ingredients such as dyes or radiopaque contrast media to aid the operator to identify the target region clearly. This can be done during the diagnosis or immediately before the ablation. A catheter containing the microwave antenna (any one of the antenna configurations (e.g. needle or conductor configurations) disclosed herein) is advanced to the target site using appropriate sheaths or scopes or visualizing techniques. The antenna is then deployed into the target tissue by penetration. Microwave energy of appropriate power and duration is then applied to denature the target tissue. The use of the coupling solution may be utilized to optimize the impedance match (and thus efficiency) in the target region and help optimize ablation of the border zone around a tumor tissue or infarcted cardiac tissue, or other treatment tissue. Additionally, the coupling solution may be utilized dynamically during energy delivery to stabilize the microwave properties of the target zone as the lesion matures. A coupling solution may be used with any of the examples of devices or methods disclosed herein.

In addition to improved efficiency via impedance matching, the coupling solution may be utilized to increase the conductivity (and absorption coefficient) of the target tissue. For example, inflated lung tissue has a conductivity of 0.79 S/m. Injection of a coupling solution such as saline into a specific target zone, with the target zone surrounded by inflated lung tissue, will increase the conductivity in that target region significantly. As a result, the target zone may undergo preferential heating due to increased absorption in that region compared with the surrounding tissue.

Microwave Antenna for Recording EGM During Ablation

It may be desirable during ablation procedures such as cardiac ablation procedures to monitor electrical activity in cardiac tissue. Performing simultaneous power delivery and electrogram recording can provide useful feedback to the operator regarding the formation of lesions during cardiac ablation.

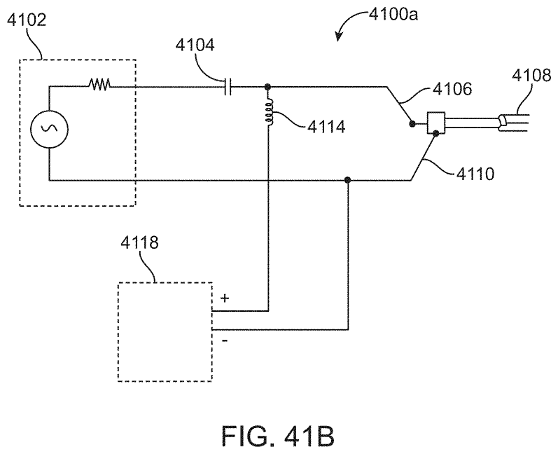

FIG. 41A shows a filter circuit 4100 that is connected to any of the applicator devices disclosed herein to allow simultaneous application of microwave energy and electrogram recording with the needles. The filter 4100 consists of two DC blocking capacitors 4104, 4116 and two Microwave Chokes 4114, 4112 (inductors). The DC blocking capacitors create a low impedance path for microwave energy and a high impedance path for low frequency cardiac electrical signals. The Microwave Chokes create a high impedance path for microwave energy and a low impedance path for low frequency cardiac electrical signals. With such a filter circuit, microwave energy that is input from a microwave generator 4102 will be transmitted into the microwave applicator 3508 with minimal leakage into the electrogram recording system. An example nominal design for operation at 2.45 GHz may have a minimum DC Blocking capacitor value of 60 pF to ensure an impedance of approximately of 1 ohm or less is presented to the microwave energy. In the same nominal design, a Microwave Choke of a minimum 68 nH inductance ensures an impedance of approximately 1000 ohms or greater is presented between the microwave generator and electrogram recording system. In the frequency range of normal cardiac electrical activity, this value of inductance also provides an impedance of less than 1 ohm between the applicator and the recording system. This nominal design will provide approximately −30 dB of isolation between the microwave generator end recording system, while having negligible microwave loss between the microwave generator and applicator. Other values of capacitance and inductance may also be possible depending on the isolation and loss requirements for the system.

FIG. 41B shows that the filter circuit in FIG. 41A can also be implemented as a single sided filter 4100a, with a DC Block and Microwave Choke 4104, 4114 only attached to the positive line 4106 of the applicator. In this configuration the negative line 4110 of the applicator is attached directly to the generator 4102 and recording system 4118.

Anchoring and Tip Stabilization

As previously discussed above with respect to FIGS. 24A-24C, any microwave ablation device disclosed herein may include some or all of the anchoring features described herein. The ablation device may include an anchor element which allows the ablation catheter to be stabilized in the tumor or other target treatment area. In an example, there may be one anchor element to stabilize the target tissue. In another example, more than one anchor elements may penetrate the target ablation region and stabilize the tumor. In any example, the one or more anchor elements penetrate the target ablation zone region before the one or more transmission lines penetrate the target ablation region. In another example, the transmission lines penetrate the target ablation region before the one or more anchor elements penetrate the target ablation region.

Any of the treatment devices disclosed herein may be delivered to the target treatment tissue in any number of ways. For example, for treating cardiac tissue, the treatment device may be disposed in a vascular catheter that can be advanced transvascularly to the treatment tissue. For treatment of lung tissue, the device may be delivered via a bronchoscope. The device may be advanced through skin into the body to treat the target tissue from outside the body, or a surgical incision may be used to provide access to the treatment tissue and the device may be advanced from outside the body through the incision. Other scopes or access routes may also be used.

### NOTES AND EXAMPLES

The following, non-limiting examples, detail certain aspects of the present subject matter to solve the challenges and provide the benefits discussed herein, among others.

Example 1 is a device for treating tissue, said device comprising: a plurality of conductors forming one or more transmission lines configured to deliver microwave energy to target tissue.

Example 2 is the device of Example 1, wherein the plurality of conductors comprises two conductors, three conductors, or four conductors.

Example 3 is the device of any of Examples 1-2, wherein the one or more transmission lines are configured such that power is reflected from an end of the transmission lines and overall reflected power decreases during at least a portion of an ablative procedure.

Example 4 is the device of any of Examples 1-3, wherein the one or more transmission lines are designed such that the reflected power decreases, reaches a minimum, and begins increasing again as ablation progresses.

Example 5 is the device of any of Examples 1-4, wherein the reflected power reaches a minimum when an ablation region is surrounded by a biocompatible solution.

Example 6 is the device of any of Examples 1-5, wherein the biocompatible solution is a normal saline solution, buffered saline solution, dextrose solution, lactate Ringer's solution, or a mixture thereof.

Example 7 is the device of any of Examples 1-6, wherein the biocompatible solution comprises radiopaque dyes, regular dyes, tissue stains, or a combination thereof to improve visualization of the target tissue.

Example 8 is the device of any of Examples 1-7, further comprising a coaxial cable having a center conductor and a shield, wherein the plurality of conductors comprise a first conductor and a second conductor, and wherein the first conductor is electrically coupled with the center conductor and the second conductor is electrically coupled with the shield.

Example 9 is the device of any of Examples 1-8, further comprising a coaxial cable having a center conductor and a shield, wherein the plurality of conductors comprise a first conductor, a second conductor, and a third conductor, and wherein the first conductor is electrically coupled with the center conductor and the second and the third conductors are electrically coupled with the shield, and wherein the first conductor is disposed between the second and third conductors.

Example 10 is the device of any of Examples 1-9, further comprising a coaxial cable having a center conductor and a shield, wherein the plurality of conductors comprise a first conductor, a second conductor, and a third conductor, and wherein the second and third conductors are electrically coupled with the center conductor and the first conductor is electrically coupled with the shield, and wherein the first conductors is disposed between the second and third conductors.

Example 11 is the device of any of Examples 1-10, wherein the plurality of conductors comprise a first conductor and a second conductor, each conductor having a proximal end and a distal end, and wherein the first and second conductors are electrically coupled together adjacent their distal ends to form an electrical short between the first and second conductors.

Example 12 is the device of any of Examples 1-11, wherein the plurality of conductors comprise a first conductor and a second conductor, and wherein the first and second conductors are coupled together to form a loop.

Example 13 is the device of any of Examples 1-12, further comprising a coaxial cable having a center conductor and a shield, wherein the plurality of conductors comprise a first conductor and a second conductor, and wherein the first conductor is electrically coupled with the center conductor and the second conductor is electrically insulated from the shield.

Example 14 is the device of any of Examples 1-13, wherein the plurality of conductors comprise a first conductor and a second conductor, and wherein the first conductor forms a loop, and wherein the second conductor is discrete from the first conductor, and the second conductor forms a second loop electromagnetically coupled with the first loop.

Example 15 is the device of any of Examples 1-14, wherein the plurality of conductors comprise a first conductor and a second conductor, and wherein the first conductor and the second conductor are discrete from one another, and wherein the second conductor is configured to be inserted into the target tissue separately from the first conductor, and wherein the first and second conductors are electromagnetically coupled with one another.

Example 16 is the device of any of Examples 1-15, wherein the plurality of conductors comprise a first conductor having a first length and a second conductor having a second length, and wherein the first length is different than the second length.

Example 17 is the device of any of Examples 1-16, further comprising a first coaxial cable having a first center conductor and a second coaxial cable having a second center conductor, and wherein the plurality of conductors comprise a first conductor coupled with the first center conductor, and a second conductor coupled with the second center conductor.

Example 18 is the device of any of Examples 1-17, wherein the plurality of conductors comprises a first plate and second plate.

Example 19 is the device of any of Examples 1-18, further comprising a printed circuit board, and wherein at least some of the plurality conductors comprise one or more traces disposed on the printed circuit board.

Example 20 is the device of any of Examples 1-19, wherein the plurality of conductors comprises a plurality of needles.

Example 21 is the device of any of Examples 1-20, wherein the plurality of needles comprise four needles disposed approximately 90 degrees circumferentially apart from one another.

Example 22 is the device of any of Examples 1-21, further comprising a temperature monitoring element.

Example 23 is the device of any of Examples 1-22, wherein at least one of the plurality of conductors comprise a fusible link that is melted or otherwise decoupled from the device by applying current therethrough.

Example 24 is the device of any of Examples 1-23, further comprising an actuatable anchor element having a collapsed configuration, and an extended configuration in which the anchor element is configured to anchor the device to the tissue.

Example 25 is the device of any of Examples 1-24, wherein the anchor element is independently advanceable and retractable relative to the plurality of conductors, and wherein the anchor element is configured to penetrate the target tissue without displacement thereof.

Example 26 is the device of any of Examples 1-25, wherein the anchor element is an active element of the one or more transmission lines.

Example 27 is the device of any of Examples 1-26, wherein the anchor element is a passive element of the one or more transmission lines.

Example 28 is the device of any of Examples 1-27, wherein at least some of the plurality of conductors are independently advanceable and retractable relative to one another.

Example 29 is the device of any of Examples 1-28, wherein at least some of the plurality of conductors are pivotable relative to one another.

Example 30 is a system for treating tissue, said system comprising the device of any of Examples 1-29; and a microwave generator.

Example 31 is the system of Example 30, wherein the microwave generator comprises a multi-frequency generator or a phase changing generator.

Example 32 is the system of any of Examples 30-31, further comprising a coaxial cable coupled to the device and a cooling element thermally coupled to the coaxial cable, and wherein the cooling element is configured to cool the coaxial cable during operation of the device.

Example 33 is the system of any of Examples 30-32, wherein the device is operably coupled to the filter circuit to allow simultaneous application of microwave energy and recording of an electrogram.

Example 34 is a method for treating tissue, said method comprising: providing an energy delivery apparatus having a plurality of conductors forming one or more transmission lines; inserting at least a portion of the plurality conductors into target tissue; delivering microwave energy to the target tissue with the plurality of conductors; and ablating the target tissue.

Example 35 is the method of Example 34, wherein delivering the microwave energy comprises delivering the microwave energy from a coaxial cable to the plurality of conductors.

Example 36 is the method of any of Examples 34-35, wherein delivering the microwave energy comprises delivering the microwave energy through the plurality of conductors, and wherein the plurality of conductors have different lengths.

Example 37 is the method of any of Examples 34-36, wherein the plurality of conductors comprises a separate conductor, and wherein inserting the plurality of conductors comprises inserting the separate conductor separately from the insertion of the other of the plurality of conductors.

Example 38 is the method of any of Examples 34-37, wherein inserting the plurality of conductors comprises disposing one or more flat plates against the target tissue.

Example 39 is the method of any of Examples 34-38, further comprising monitoring a temperature of the target tissue.

Example 40 is the method of any of Examples 34-39, further comprising passing a current through at least one of the plurality of conductors and melting the at least one of the plurality of conductors or otherwise separating the at least one of the plurality of conductors into a plurality of segments.

Example 41 is the method of any of Examples 34-40, wherein delivering the microwave energy comprises delivering multiple frequencies or multiple phases of microwave energy.

Example 42 is the method of any of Examples 34-41, further comprising cooling the energy delivery apparatus or a coaxial cable coupled thereto.

Example 43 is the method of any of Examples 34-42, further comprising actuating an anchor element on the energy delivery apparatus and anchoring the energy delivery apparatus to the target tissue or tissue adjacent thereto.

Example 44 is the method of any of Examples 34-43, wherein the anchoring is achieved prior to inserting the plurality conductors into the target tissue.

Example 45 is the method of any of Examples 34-44, further comprising advancing or retracting at least some of the plurality of conductors independently of one another.

Example 46 is the method of any of Examples 34-45, further comprising pivoting at least some of the plurality of conductors relative to one another.

Example 47 is the method of any of Examples 34-46, further comprising injecting a biocompatible solution into the target tissue and altering a property of the target tissue to facilitate microwave ablation of the target tissue, or to facilitate visualization of the target tissue.

Example 48 is the method of any of Examples 34-47, wherein injecting the biocompatible solution occurs during ablation of the target tissue.

Example 49 is the method of any of Examples 34-48, wherein injecting the biocompatible solution occurs before ablation of the target tissue.

In Example 50, the apparatuses or methods of any one or any combination of Examples 1-49 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A device for treating tissue, said device comprising:
a plurality of conductors forming one or more transmission lines configured to deliver microwave energy to target tissue.

2. (canceled)

3. The device of claim 1, wherein the one or more transmission lines are configured such that power is reflected from an end of the transmission lines and total reflected power decreases during at least a portion of an ablative procedure.

4. (canceled)

5. (canceled)

6. (canceled)

7. (canceled)

8. The device of claim 1, further comprising a coaxial cable having a center conductor and a shield, wherein the plurality of conductors comprise a first conductor and a second conductor, and wherein the first conductor is electrically coupled with the center conductor and the second conductor is electrically coupled with the shield.

9. (canceled)

10. (canceled)

11. The device of claim 1, wherein the plurality of conductors comprise a first conductor and a second conductor, each conductor having a proximal end and a distal end, and wherein the first and second conductors are electrically coupled together adjacent their distal ends to form an electrical short between the first and second conductors.

12. (canceled)

13. The device of claim 1, further comprising a coaxial cable having a center conductor and a shield, wherein the plurality of conductors comprise a first conductor and a second conductor, and wherein the first conductor is electrically coupled with the center conductor and the second conductor is electrically insulated from the shield.

14. (canceled)

15. The device of claim 1, wherein the plurality of conductors comprise a first conductor and a second conductor, and wherein the first conductor and the second conductor are discrete from one another, and wherein the second conductor is configured to be inserted into the target tissue separately from the first conductor, and wherein the first and second conductors are electromagnetically coupled with one another.

16. (canceled)

17. (canceled)

18. (canceled)

19. (canceled)

20. The device of claim 1, wherein the plurality of conductors comprises a plurality of needles.

21. The device of claim 20, wherein the plurality of needles comprise four needles disposed approximately 90 degrees circumferentially apart from one another.

22. The device of claim 1, further comprising a temperature monitoring element.

23. The device of claim 1, wherein at least one of the plurality of conductors comprise a fusible link that is melted or otherwise decoupled from the device by applying current therethrough.

24. The device of claim 1, further comprising an actuatable anchor element having a collapsed configuration, and an extended configuration in which the anchor element is configured to anchor the device to the tissue.

25. (canceled)

26. (canceled)

27. (canceled)

28. The device of claim 1, wherein at least some of the plurality of conductors are independently advanceable and retractable relative to one another.

29. (canceled)

30. A system for treating tissue, said system comprising:
the device of claim 1; and
a microwave generator.

31. (canceled)

32. The system of claim 30, further comprising a coaxial cable coupled to the device and a cooling element thermally coupled to the coaxial cable, and wherein the cooling element is configured to cool the coaxial cable during operation of the device.

33. The system of claim 30, further comprising a filter circuit, wherein the device is operably coupled to the filter circuit to allow simultaneous application of microwave energy and recording of an electrogram.

34. A method for treating tissue, said method comprising:
providing an energy delivery apparatus having a plurality of conductors forming one or more transmission lines;
inserting at least a portion of the plurality conductors into target tissue;
delivering microwave energy to the target tissue with the plurality of conductors; and
ablating the target tissue.

35. (canceled)

36. (canceled)

37. The method of claim 34, wherein the plurality of conductors comprises a separate conductor, and wherein inserting the plurality of conductors comprises inserting the separate conductor separately from the insertion of the other of the plurality of conductors.

38. (canceled)

39. The method of claim 34, further comprising monitoring a temperature of the target tissue.

40. The method of claim 34, further comprising passing a current through at least one of the plurality of conductors and melting the at least one of the plurality of conductors or otherwise separating the at least one of the plurality of conductors into a plurality of segments.

41. (canceled)

42. The method of claim 34, further comprising cooling the energy delivery apparatus or a coaxial cable coupled thereto.

43. The method of claim 34, further comprising actuating an anchor element on the energy delivery apparatus and anchoring the energy delivery apparatus to the target tissue or tissue adjacent thereto.

44. (canceled)

45. (canceled)

46. (canceled)

47. The method of claim 34, further comprising injecting a biocompatible solution into the target tissue and altering a property of the target tissue to facilitate microwave ablation of the target tissue, or to facilitate visualization of the target tissue.

48. (canceled)

49. (canceled)
